(19) United States
(12) Patent Application Publication
(10) Pub. No.: US 2009/0170059 A1
(43) Pub. Date: Jul. 2, 2009

(54) Methods for Preparing Cord Matrix Stem Cells (CMSC) for Long Term Storage and for Preparing a Segment of umbilical cord for cryopreservation

(76) Inventor: Hans Klingemann, Boston, MA (US)

Correspondence Address: LAWSON & WEITZEN, LLP, 88 BLACK FALCON AVE, SUITE 345, BOSTON, MA, 02210, US

(21) Appl. No.: 12/084,931

(22) PCT Filed: Nov. 14, 2006
(86) PCT No.: PCT/US2006/044094

§ 371 Date: May 13, 2008

Related U.S. Application Data
(60) Provisional application No. 60/736,058, filed on Nov. 14, 2005.
(60) Provisional application No. 60/749,523, filed on Dec. 13, 2005.

(51) Int. Cl. A01N 1/02; C12N 5/06; C12N 5/08; C12N 15/87

(52) U.S. Cl. 435/1.3; 435/374; 435/366; 435/455

(57) ABSTRACT

Methods and kits are provided for preparation of umbilical cord fragments and cells using autologous blood or blood products, and for storage of these materials with autologous cells and blood or blood products in containers having a plurality of separable chambers.

### FIELD OF THE INVENTION

Methods and kits for preparing cord matrix stem cells for cryopreservation are provided.

### BACKGROUND

Stem cells are considered potentially useful for treatment of a large variety of human and animal conditions, for example, replacement and repair of tissues such as pancreatic islets, severed nerve cells, skin grafts for burns or abrasions, and hematopoietic cells following chemotherapy and radiation. Cells obtained from various sources, for example, embryonic stem cells, placenta stem cells, amniotic stem cells, cord blood stem cells, cord matrix stem cells and other forms of adult stem cells generally have ability to differentiate into a variety of tissue types and potentially entire organs.

Although embryonic stem cells hold promise for tissue and organ generation, stem cells with early mesenchymal character, which are obtained at the time of birth from extra-embryonic tissue, may have similar capabilities if manipulated appropriately. These “peri-natal” tissues such as the umbilical cord and placenta structures, which are generally discarded after delivery, contain early mesenchymal stem cells that are believed to have a greater potential for plasticity than post-natal mesenchymal cells. Early mesenchymal cells express early transcriptional genes, and as emerging technologies such as nuclear reprogramming could direct their development into tissues of embryonic origin, these cells, generally discarded after birth, could become a valuable source for future tissue generation.

Umbilical cord blood (UCB) is a rich source of biological materials including cells such as hematopoietic stem cells, and is readily available from placenta following childbirth. Public cord blood banks have been established in the United States and abroad to collect, process and store UCB for use in transplantation. To date, more than 3000 UCB transplants have been performed in children and adults around the world (Kurtzberg J et al., N Engl J Med 335: 157, 1996; Gluckman E et al., Exp Hematol 32: 397, 2004; Gluckman E et al., Rev Clin Exp Hematol 5: 87, 2001; Laughlin M J et al., N Engl J Med 344: 1815, 2001; and Barker J N et al., Blood 105: 1343, 2005), used to treat patients with leukemia and lymphoma. Cord blood is a stem cell source for those patients who do not have a sibling donor, or cannot wait for a long search to find a matched marrow donor. UCB cells induce less incidence of graft versus host disease than blood or marrow stem cells and hence allow transplantation across HLA barriers commonly found among human populations.

Marrow stromal cells compose a heterogenous population, and include: reticular endothelial cells, fibroblasts, adipocytes, and osteogeneic precursor cells, which provide growth factors, cell-to-cell interactions, and matrix proteins, which are derived from common precursor cells that reside in the bone marrow microenvironment and are referred to as mesenchymal stem cells (MSC; Pittenger M F et al., Science 284: 143, 1999; and Muraglia A et al., J Cell Sci 113: 1161, 2000). Similar cells have been found in the lung (in't Anker P S et al., Exp Hematol 31: 881, 2003), in UCB (Noort W A et al., Exp Hematol 30: 870, 2002) and in the placenta (Li, C et al., Exp Hematol 32: 657, 2004). MSC can be distinguished from hematopoietic stem cells based on a lack of CD34 expression, and are negative also for CD45, and are positive for CD73, CD105 and MHC class I antigens. MSC exhibit multilineage differentiation capacity and are able to generate progenitors with more restricted development potential, including fibroblasts, osteoblasts, and chondrocyte progenitors (Pittenger M F, et al., Science 284: 143, 1999; and Muraglia A et al., J Cell Sci 113: 1161, 2000), and are able to generate a variety of differentiated cell types, for example, those found in embryonic germ layers, such as bone, cartilage, fat, tendon, muscle, marrow stroma and even cardiomyocytes.

Early pluri-potential MSC from peri-natal tissues such as the umbilical cord (Wang H-S, Stem Cells 22:1330-1337, 2004), placenta (Zhang Y et al, Exp Hematol 32:657-664, 2004) amnion (Miki T, Stem Cells 23:1549-1559, 2005) or even cord blood (Koegler G et al., Exp Hematol 33: 573-583, 2005) may contain stem cells that could be manipulated either by external factors or at the gene level to develop into different cell types that can be used for tissue generation similar to or instead of embryonic stem cells.

Cord matrix stem cells (CMSC) are mesenchymal-like cells that are located in the circumference of the umbilical cord. CMSC express characteristic surface markers (CD44, CD73, CD105) and integrin markers (CD29, CD51), and lack certain hematopoietic lineage markers (CD34 and CD45).

Culture or cryopreservation of cells in the presence of serum or plasma that is xenogeneic (i.e. fetal calf or fetal bovine serum or plasma), or even allogeneic, changes the pattern of expression of genes, in addition to inducing an immune response. Addition of fetal calf or bovine serum or plasma to CMSC was found to induce an unstable transcriptional profile (Shahdadfar A et al., Stem Cells 23: 1357, 2005) and lead to over-expression of collagen, changing the adherence characteristics of the cells. Thus, cells contacted with a xenogeneic or allogeneic serum or plasma display significantly different cell expression profiles from cells prior to this process, and are substantially altered physiologically, functionally, and even genetically, as a result of contact with allogeneic or xenogeneic materials. See U.S. patent application publication numbers 2003/0161818; 2005/0148074; and 2005/0054098.

There is a need for a method of isolating and cryopreserving CMSC and cells from UCB under current good manufacturing practices (cGMP) and current good tissue practices (cGTP), and under conditions that do not affect the biological characteristics of the cells for use for therapeutic purposes.

### SUMMARY

The invention in one embodiment provides a method for preparing cord matrix stem cells (CMSC) for cryopreserving, the method including steps of contacting the CMSC with a cryoprotectant and cord blood serum or plasma, wherein the serum or plasma is obtained from a source autologous in origin to the CMSC. The cryoprotectant is chosen from, for example, dimethyl sulfoxide, glycerol, ethylene glycol, or propylene glycol.

In a related embodiment, CMSC are isolated from a plurality of locations along an entire circumference of a transverse section of an umbilical cord. In a related embodiment, the source is human.

In another related embodiment, after obtaining the CMSC from the source, the CMSC are cryopreserved without culturing the cells to expand the cell number.

In an alternative embodiment, prior to cryopreserving, the CMSC cell number is expanding by culturing. Expanding the CMSC includes culturing the cells, for example, for at least one day, for example, or for at least two days.

In another related embodiment, obtaining the CMSC further includes, prior to cryopreserving, dissecting the cord to obtained resulting fragments, and isolating the CMSC from the fragments. Alternatively, the fragments are cryopreserved prior to isolating the CMSC.

In general, cord, blood and/or plasma are contacted using sterile technique, sterile apparatus, and sterile buffers, wherein the buffers are adjusted to physiological pH and osmolarity.

Another embodiment of the invention provided herein is a method of cryopreserving, separately or together, a plurality of types of stem cells from a subject, the method including steps of apportioning the types of stem cells into a separate chamber of a container comprising a plurality of chambers, wherein each of the chambers is separately accessible. The container is in one embodiment a plastic bag, and the separated chambers are separable compartments of the bag. The types of stem cells are obtained from sources including cord, matrix, placenta, cord matrix stem cells (CMSC) and blood cells.

Another embodiment of the invention provided herein is a method of preparing an umbilical cord obtained from an animal subject for cryopreservation, the method including steps of: preparing a plurality of segments of the cord; dissecting each of the plurality of segments, wherein a plurality of resulting cord fragment preparations are obtained from each of the segments; and cryopreserving separately each of the plurality of fragment preparations, wherein the umbilical cord is cryopreserved. In one embodiment, the segments are less than about 2 cm in length. In an alternative embodiment, the segments are less than about 1 cm in length.

In another embodiment, cord matrix stem cells (CMSC) are isolated from the fragments after cryopreserving. In general, the cord is contacted with sterile plasticware or glassware, and sterile buffer of physiological pH and osmolarity prior to dissecting.

In another embodiment, the segments are taken from all or a portion of a circumferential transverse section of the cord.

Another embodiment of the invention provided herein is a kit including a plurality of chambers such that each chamber contains at least one cryopreserved material selected from the group of cord matrix stem cells (CMSC) and cord blood cells, and the CMSC and cord blood cells are obtained from an autologous source, and the chambers comprise separate compartments attached within a container, each chamber separably accessible so that within each chamber are provided independently with respect to the remainder of the chambers. In a related embodiment, each chamber contains a unit dose of CMSC.

Another embodiment of the invention provided herein is a kit that has a plurality of chambers each including cryopreserved cord matrix stem cells (CMSC) and cord blood cells, such that the chambers have separate compartments that are attached and are within a plastic bag, and the CMSC and cord blood cells within each chamber are autologous, such that each chamber is separately openable and CMSC within each chamber are used independently with respect to the remainder of the chambers. In a related embodiment, each chamber contains a unit dose of CMSC. In another related embodiment, the CMSC and/or cord blood cells in the plurality of chambers are from an autologous source.

Another embodiment of the invention provided herein is a method of increasing the number of hematopoietic cells, the method including transfecting at least one gene into feeder cells thus improving ability of the feeder cells to serve as a feeder layer; and culturing the hematopoietic cells with the feeder layer, so that the number of hematopoietic cells is increased. In a related embodiment of the method, culturing further includes using a blood product that is autologous to the hematopoietic cells or the feeder cells.

In yet another related embodiment of the method, the gene encodes at least one protein selected from the group consisting of granulocyte-colony stimulating factor (G-CSF), granulocyte macrophage cell stimulating factor (GM-CSF), stem cell factor (SCF), thrombopoietin (TPO), erythropoietin (EPO), epidermal growth factor (EGF), keritinocyte growth factor (KGF), and other proteins that support the expansion and proliferation of cells.

In still another related embodiment of the method, the feeder cells are Wharton's Jelly cells. In another related embodiment, the hematopoietic cells are CD34⁺ hematopoietic progenitor cells. In another related embodiment, the method further includes culturing the CD34⁺ hematopoietic progenitor cell and developing the cells into least one cell type selected from the group consisting of natural killer cells, T cells, and dendritic cells. In still another related embodiment of the method, the hematopoietic cells and the feeder cells are autologous.

Another embodiment of the invention provided herein is a method of preparing feeder cells, the method including, genetically manipulating feeder cells, such that the genetic manipulating results in improving an ability of the feeder cells to serve as a feeder layer. In a related embodiment of the method, the feeder cells are genetically manipulated by transfecting genes into the feeder cells encoding at least one of granulocyte-colony stimulating factor (G-CSF), granulocyte macrophage cell stimulating factor (GM-CSF), stem cell factor (SCF), thrombopoietin (TPO), erythropoietin (EPO), EGF, KGF, and other proteins that support the expansion and proliferation of cells.

In another related embodiment of the method, prior to manipulating, the method includes isolating the feeder cells from human umbilical cord. In yet another related embodiment of the method, isolating the feeder cells involves obtaining Wharton's Jelly cells.

### BRIEF DESCRIPTION OF THE DRAWINGS

FIG. 1 is a photomicrograph of a cross-section of an umbilical cord.

FIG. 2 is a drawing of a multi-chambered container.

FIG. 3 is a photomicrograph of CMSC.

FIG. 4 is a set of graphs showing flow cytometric profiles of CMSC.

### DETAILED DESCRIPTION

Because of potential uses of CMSC for therapeutic purposes in humans or other mammals, there is a need in the art for a method of isolating and cryopreserving these cells in compliance with cGMP and cGTP standards and for further methods that do not change the relevant biological characteristics of these cells. Prior art methods pertaining to cells obtained from umbilical cord do not address the issues of storing CMSC under conditions that conform to FDA standards and that maintain their biologic characteristics.

Umbilical cord blood (UCB) stem cells provide a readily available source for hematopoietic stem cells. UCB has a number of proven advantages as a source of hematopoietic stem cells for transplantation. One advantage is that UCB is an abundantly available source of stem cells that is currently discarded and can be harvested at no risk to the mother or infant. In contrast, in bone marrow and peripheral blood donations there is a risk imposed on the donor associated with the procedure, in addition to the inconvenience.

Another advantage of UCB is that major infectious agents, such as cytomegalovirus (CMV), are much less common in the newborn than adults, and are less likely to contaminate UCB. UCB units, typed, cryopreserved and banked, also are available on demand, eliminating delays and uncertainties that complicate marrow collection from unrelated donors. At present, UCB can be delivered for infusion within days of initiation of a search. This compares with a median of 3-4 months from search to delivery of stem cells through registries of volunteer adult donors. Frozen UCB also can be easily shipped, stored at the treating institution, and thawed for use when needed, compared to freshly donated bone marrow which has a limited shelf-life of one day or less, necessitating coordination between harvesting surgeons, transportation, and transplantation teams.

A further advantage of UCB as a source of stem cells is that the intensity of graft-versus-host reactivity of fetal lymphocytes appears to be less than that of adult cells and consequently fetal lymphocytes are more tolerant of HLA incompatibility. Published studies have shown that transplantation of UCB matched at 4 to 5 out of 6 antigens results in a similar incidence of GvHD to transplantation of unrelated bone marrow fully matched at 6 out of 6 antigens (Gluckman E et al., Exp Hematol 32: 397, 2004; Gluckman E et al., Rev Clin Exp Hematol 5: 87, 2001; and Laughlin M J et al., N Engl J Med 344: 1815, 2001). However, extent of engraftment of cells over a prolonged period of time continues to be a problem accounting for morbidity and mortality. At present, shortening of the engraftment period is achieved by providing sufficient numbers of UCB cells, which restricts the recipient pool to children and small adults.

Although research is ongoing to obtain methods that expand ex vivo the number of available UCB stem cells, these approaches have resulted in the expansion primarily of committed progenitor cells, with no significant beneficial impact on the time of bone marrow recovery. Efforts to accelerate the pace of engraftment via ex vivo expansion of UCB units have not improved clinical outcomes (Gluckman E et al., Rev Clin Exp Hematol 5: 87, 2001; and Laughlin M J et al., N Engl J Med 344: 1815, 2001). Evidence in both animal models and human studies suggests that methods utilizing cytokines such as granulocyte-colony stimulating factor (G-CSF), stem cell factor (SCF), and thrombopoietin (TPO) in liquid cultures expand predominantly short-term committed hematopoietic progenitors, at the expense of long-term progenitors, which are the cells that will lead to sustained hematopoiesis (Williams D A, Blood 81: 3169, 1993; McNiece I K, Exp Hematol 30: 612, 2002; Von Drygalski A et al., Stem Cells Dev 13: 101, 2004; and Tisdale J F et al., Blood 92: 1131, 1998). However, another disadvantage hampering the exploration of ex vivo stem cell expansion approach is the availability of clinical growth factors. In addition, the majority of cord blood banks preserve only a single unit of frozen material from a donor source. Clinical trials have typically expanded only a fraction (10-60%) of the frozen cells, with the remainder infused unmanipulated.

Ex vivo expansion of cord blood stem cells is accomplished by using bone marrow derived MCS as a feeder layer (Robinson S N et al., Bone Marrow Transplant 37: 359, 2006). MSC were generated from adult bone marrow, and when serving as a monolayer platform for UCB cells together with cytokines (usually a combination of an interleukin such as IL-3, IL-6, and with G-CSF, SCF, FLT-3L, EPO), resulted in faster engraftment. Flow immunocytometric analysis shows that mice that received UBC cells expanded by culture on a layer of MSC had about three times as many human cells (CD45 positive) in the marrow after the transplant, than mice that received an infusion of uncultured cells (Kadereit S et al., Stem Cells 20: 573, 2002). Stroma contact of hematopoietic stem cells was found to be superior to culture in cytokine supplemented (McNiece I et al., Cytotherapy, 6: 311, 2004; Yildirim S et al., Bone Marrow Transplant 36: 71, 2005; Breems D A et al., Blood 91: 111, 1998; Zhang Y et al., Exp Hematol 32: 657, 2004; and Kanai M et al., Bone Marrow Transplant 26: 837, 2000).

To solve the problem of long engraftment, another approach taken in animal models is co-injection of MSC with UBC. Thus mice were administered non culture-expanded fetal lung-derived CD34 negative MSC (in't Anker PS et al., Exp Hematol 31: 881, 2003). Results showed that transplantation of a mixture of human UCB CD34⁺ cells (at either of four concentrations, 0.03, 0.1, 0.3, and 1×10⁶) in the presence of MSC (10⁶) resulted in significantly faster engraftment in bone marrow of NOD/SCID mice, than that observed after transplantation with control UCB CD34⁺ cells alone (n=22 versus 29 days, p<0.05). The most pronounced effect on bone marrow engraftment was observed after transplantation of relatively low doses of CD34⁺ UCB cells (0.03-0.1×10⁶). Co-transplantation of MSC resulted in a three-fold to four-fold increase in the percentage of human CD45⁺ cells in the bone marrow (14% versus 4.7% at 0.03×10⁶ cells, and 40% versus 10% at 0.1×10⁶ CD34⁺ cells, p<0.001). However, the majority of the infused lung-derived MSC were observed in the lung and not in the bone marrow. Improved engraftment was observed when a lower number of CD34⁺ cells (<1×10⁶) were infused into irradiated NOD/SCID mice infused with a mixture of human mobilized peripheral blood hematopoietic stem cells and culture-expanded MSC harvested from adult bone marrow (Angelopoulou M et al., Exp Hematol 31: 413, 2003). See also in't Anker P S et al., Exp Hematol 31: 881, 2003. Further, co-transplantation of human stromal cells into pre-immune sheep supported faster recovery after marrow transplant (Maitra B et al., Bone Marrow Transplant 33: 597, 2004). Clinical feasibility has been shown by co-transplanting culture expanded HLA identical mesenchymal stem cells with marrow stem cells in patients with hematopoietic malignancies (Lazarus H M et al., Biol Blood Marrow Transplant 11: 389, 2005).

An additional method of potentially enhancing engraftment of a suboptimal dose of UCB cells is direct intraosseous infusion, or intra-bone marrow transplant. Bone marrow transplant directly into bone was shown long ago, however this procedure was abandoned for its morbidity, especially after discovery that intravenous infusion yielded comparable or superior results (Kadereit S et al., Stem Cells 20: 573, 2002). Stem cells are known to transit intravenously through various organs before reaching their final destination in the bone marrow, however up to 90% of infused hematopoietic stem cells will lodge in the lungs. Investigators have therefore re-considered injecting stem cells directly into the marrow space. Stem cells are directly inserted into the bone marrow microenvironment, which is known to contain molecular cues to direct hematopoiesis. Studies in mice have shown that this approach results in faster engraftment and long-term engraftment of the injected stem cells (Levac K et al., Exp Hematol. 33: 1417, 2005; and Wang J et al., Blood 101: 2924, 2003). Initial clinical trials have injected bone marrow cells into the pelvis. Surprisingly, volumes as large as one liter were tolerated without significant side effects (Hagglund H et al., Bone Marrow Transplant 21: 331, 1998). Although no benefit was seen with respect to shortening of the engraftment time, these studies were not designed to analyze such a benefit, as patients received a full marrow transplant in a conventional way at the same time, representing sufficient numbers of stem cells to guarantee timely engraftment.

It is here envisioned that UCB cells may be utilized as a stem cell source in this setting, and it is here further envisioned that co-infusion with MSC into the marrow along with umbilical cord blood could lead to enhanced engraftment. Providing injection of UCB cells directly into the marrow to accelerate engraftment would allow a transplant to be performed with suboptimal umbilical cord blood stem cell numbers.

Examples herein use co-transplantation of umbilical cord matrix (UCM) cells, a type of mesenchymal cell that is obtained from the Wharton's Jelly of the umbilical cord, to support faster engraftment of UCB cells and thereby facilitate transplantation into recipients that are larger adults. These cells optimize UCB cell homing and blood cell production, under conditions where only limited numbers of UCB cells have been transplanted.

Expression of genes found in early development and required for self renewal and pluripotency, such as Oct-4 and nanoc, was observed in MSC obtained from peri-natal tissues, materials that are usually discarded after birth, such as the umbilical cord, placenta, amnion, and chorion (Wang H-S, Stem Cells 22:1330-1337, 2004; Zhang Y et al, Exp Hematol 32:657-664, 2004; Miki T, Stem Cells 23:1549-1559, 2005; and Koegler G et al., Stem Cells 33: 573-583, 2005). Further, MSC express genes associated with each of the three principal germinal layers: ectoderm, mesoderm and endoderm, and are presumably in a state of transition to the mesenchyme found at a later development state in bone marrow. Without being limited by any particular mechanism or theory, those genes could be manipulated and activated in a method causing the cells to differentiate along each of a plurality of cell lineages.

For example, it is here envisioned that early MSC can be programmed to develop into insulin secreting cells. Alternatively, peri-natal MSC have the potential for use to improve engraftment after bone marrow and stem cell transplant. Delayed engraftment can be a significant problem, especially after cord blood transplant. Co-transplantation of cord blood cells together with peri-natal MSC speeds engraftment and facilitates transplantation, particularly in transfusions where only limited numbers of hematopoietic stem cells are available. The MSC that make up the bone marrow stroma can provide an essential structural network for hematopoietic stem cells in addition to producing cytokines that support their maturation and differentiation. MSC can also be used for down-regulating the immune response using bone marrow derived MSC in autoimmune diseases and graft-versus-host disease after bone marrow transplantation.

Different types of mesenchymal-like cells have been from isolated from umbilical cords, for example, by a method in which vessels of the umbilical cord are first removed and discarded to harvest the remaining tissue, known as Wharton's Jelly (Mitchell et al., Stem Cells 21: 50-60, 2003). Peri-natal MSC, and in particular MSC from the umbilical cord, can easily be obtained after delivery. A small amount of cord tissue provides sufficient cells for expansion, and can be frozen and stored along with cord blood of a newborn. Cells can be thawed and processed when needed at a later point. Such cells provided by the methods herein are advantageous because they are autologous and therefore carry no risk of rejection.

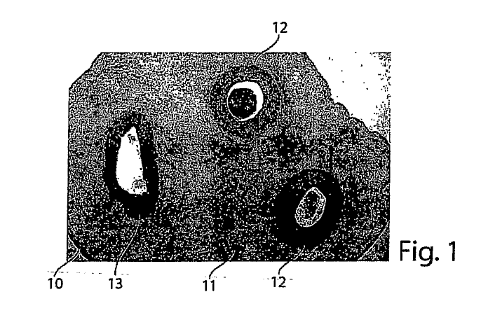

A cross-section of an umbilical cord 10 is shown in FIG. 1. A majority of the tissue in the cord consists of the Wharton's Jelly 11, which surrounds the umbilical veins 12 and artery 13. Wharton's Jelly includes connective tissue of the umbilical cord, a mixture of a gelatinous intercellular substance, collagen fibers, hyaluronic acid, and cells such as myofibroblasts and fibroblasts. The Wharton's Jelly mixture acts as a physical buffer, preventing kinking of the umbilical cord and thereby preventing disruption of maternal-fetal circulation (Sackier et al., U.S. Pat. No. 5,612,028). It is here proposed that Wharton's Jelly cells are very early stem cells. The first “blood islands” or developmental site of hematopoiesis is the extraembryonic yolk sac followed by the aortic-gonad-mesonephros (AGM). The region is thought to produce populations of mesenchymal cells, vascular progenitors and perhaps hemangioblasts. From the AGM region there is a migration of precursors to the fetal liver through the allantois. During or shortly after this migration a portion of these multipotential progenitors are trapped in the Wharton's Jelly of the developing placenta and umbilical cord.

A limiting factor in commercial development of cord blood transplant is the low number of hematopoietic stem cells, which can lead to delayed engraftment and decreased survival. The cord stem cell number frequently is insufficient to transplant adult patients. Examples herein show methods to isolate a type of ‘support’ cells from the Wharton's Jelly of the umbilical cord. Examples show that Wharton's Jelly cells can increase the number and function of blood-forming stem cells. Mice are simultaneously transplanted with cord blood cells with Jelly cells, to show faster engraftment and allow to transplant adults for whom there are not enough stem cells in the cord blood.

Cord blood transplants are done worldwide, mostly in children and small adults, as the number of stem cells in the banked units is frequently too low to support timely engraftment in larger adults. Stem cells currently are not present in the cord blood in sufficient amounts to support hematopoiesis in larger individuals, limiting more widespread use of cord blood cells for transplantation in adults. Even with optimization of the collection process, the majority of collections are not sufficient for larger adults. Attempts to expand cord blood cells ex vivo in a cytokine cocktail have met with only limited success. The volume of cryopreserved cord blood units and the large body size of most adult patients limit the dose of cells (number of cells per kilogram of body weight) that can be infused to establish donor hematopoiesis. Limited cell doses lead to prolonged engraftment times, increased risk of engraftment failure and consequent increased risks to patients.

Transplant centers therefore have certain guidelines in place that define a minimum number of cells. Single units for infusion generally have a cryopreserved cell dose greater than 2.0×10⁷ mononuclear cells (MNC) per kilogram of recipient body weight. Thus there is a probability of only 4% of finding a transplantable cord blood unit in the current registries, of sufficient size for for a 70 kg adult, compared to 94% probability for a 10 kg child. (Thomas E D, Int J Hematol 81: 89, 2005).

Even if a cell dose of more than 2.0×10⁷ MNC/kg is transplanted, the median time to recover more than 500/mm³ neutrophils is 25 days and 59 days to achieve a platelet count of 20,000/mm³ (Kurtzberg J et al., N Engl J Med 335: 157 (1996); Gluckman E et al., Exp Hematol 32: 397, 2004; Gluckman E et al., Rev Clin Exp Hematol 5: 87, 2001; Laughlin M J et al., N Engl J Med 344: 1815, 2001; and Barker J N, et al., Blood 105: 1343, 2005). This is a median value, suggesting that 50% of patients will take even longer for their marrow to recover. This prolonged marrow recovery increases the risk of infections as well as costs related to blood and platelet support, extended hospitalization and frequent hospital readmissions. The transplantation of two unrelated cord blood units has shown some shortening of engraftment but this effect is not dramatic and carries a higher costs. Rates of acute GvHD are similar to those reported for matched unrelated transplant allogeneic transplant (Kurtzberg J et al., N Engl J Med 335: 157, 1996; Gluckman E et al., Exp Hematol 32: 397, 2004; Gluckman E et al., Rev Clin Exp Hematol 5: 87, 2001; Laughlin M J et al., N Engl J Med 344:1815, 2001; and Barker J N, et al., Blood 105: 1343, 2005). Most recipients of cord blood units are mismatched at one or two of the six HLA loci (i.e., each of loci HLA-A, HLA-B, and HLA-DR on each of two paired chromosomes). Such HLA antigen incompatibility in matched unrelated transplants is associated with poor outcomes, due to graft failure and GvHD. Tolerance of HLA-incompatibility by a cord blood graft makes cord blood valuable as a stem cell source. Even within the relatively small pool of banked cord units, matching a minimum of four or five antigens instead of all six greatly increases the likelihood that a match will be found.

Wharton's Jelly or umbilical cord matrix represents a rich source of primitive multipotent MSC like progenitor cells which are currently not widely appreciated as a source of MSC. MSC cells were characterized by several investigators (Eyden, J Submicrosc Cytology 26: 347, 1994; Wang H S et al., Stem Cells 22: 1330, 2004; Weiss M L, et al., Stem Cells 24: 781-792, 2006; Weiss M L et al., Exp Neurol 182: 288, 2003; Fu Y S et al., J Biomed Sci. 11: 652, 2004; Fu Y S et al., Stem Cells 24: 115, 2005; Sarugaser R et al., Stem Cells 23: 220, 2005; and Carlin R et al., Reprod Biol Endocrinol 4: 8, 2006). MSC from adult bone marrow are rare (less than 0.001% of cells) and must be harvested from adult volunteers. The cells do not appear to be immortal, leading researchers to search for more viable sources of MSC that potentially could support cord blood cell engraftment. A MSC-like cell has been isolated from the UCB and termed an unrestricted somatic stem cell (US SC) (Kogler G. et al., Exp Hematol 33: 573, 2005). However the recovery of those cells is relatively low and only one third of fresh cord blood specimens will yield USSC upon culture.

Cells from cord have a much longer life span than bone marrow derived MSC, and express the transcription factors Oct-4 and nanog that are important for maintaining an undifferentiated state and pluripotent capacity. The Wharton's Jelly contains a large amount of early MSC that are obtained from cord which is otherwise discarded after delivery (Koegler G et al., Exp Hematol 33: 573-583, 2005). These cells display pluri-potent capacity, with potential applications such as use in spinal cord injuries, to accelerate wound healing or to treat Parkinson's disease (Weiss M L et al., Stem Cells 24: 781-792, 2006). Beyond using these early pluripotential MSC merely for regenerative medicine, they may be used also as carriers of targeted molecules, cytokines and drugs. Examples are molecules that increase angiogenesis or prevent scarring and fibrosis. Peri-natal MSC can easily be transduced and can be used as vehicles for either short-term or long-term expression of genes of interest.

Since MSC are known to target sites of inflammation, and cancerous cells generally initiate a state of inflammation around them, MSC migrate to tumor sites. MSC obtained from bone marrow and transfected with an interferon gene have been shown in a murine model to travel to malignant sites and release a cytokine locally, resulting in an anti-tumor effect (Deans R J et al., Exp Hematol 28: 875-884, 2000). MSC can be used as biological pumps to inhibit degenerative and support restorative events. Genetic manipulation of these cells extends the life span. Even without manipulation, these cells are capable of at least twice as many doublings as MSC obtained from bone marrow that is more mature. In addition, peri-natal MSC having low immunogenicity are useful as allogeneic donor cells, to establish cell lines for further manipulation.

Peri-natal MSC are here envisioned to have a further role in generating more complex tissues, for which certain scaffolds such as bone and vessels are supplied. Alternatively the cells may serve as vehicles for delivery of site directed morphogenic proteins. Peri-natal cells per se take on features of embryonic stem cells by ‘nuclear reprogramming’ at the genetic level (Dembinski J L et al., Cytotherapy 888, 2006). Certain progenitor cells remain responsive to embryonic transcription factors (Hochedlinger K et al., Nature 441:1061-1067, 2006). Somatic cells regress when the transcription factor Oct-4 is turned off. As Oct-4 is expressed in peri-natal MSC from extra-embryonic tissue, reprogramming MSC is envisioned herein to involve turn-off of Oct-4. MSC are used to provide the framework (stroma) so that tissue specific stem cells of multi-potential capacity differentiate into a fully functional tissue.

Mesenchymal stem cell-like cells surrounding the vasculature of the cord have been isolated from the umbilical cord (Romanov et al., Stem Cells 21: 105-110, 2003). Collagenase digestion from within the umbilical vein has been used to obtain a mixed population of vascular endothelial and sub-endothelial cells.

A procedure to collect Wharton's Jelly from the umbilical cord under sterile conditions is shown in U.S. patent application publication number 2003/0161818. In this procedure, the cord is cut transversely with a scalpel, and each section is transferred to a sterile container containing phosphate buffered saline (PBS) with CaCl₂ (0.1 g/l) and MgCl₂6H₂O (0.1 g/l) to remove surface blood from the section with gentle agitation. The section is then removed to a sterile-surface where the outer layer of the section is incised along the longitudinal axis of the cord, and blood vessels of the umbilical cord (two veins and an artery) are removed by dissection, for example, with sterile forceps and dissecting scissors. Wharton's Jelly is collected into a sterile container, or cut into smaller sections, of size such as 2-3 mm³ for culturing the included cells.

Umbilical cord matrix (UCM) cells express CD44, CD29, CD51 and not hematopoietic lineage markers (CD34, CD45, CD3, CD5, CD14, CD19). Further, UCM express MSC markers (SH2 also known as CD105, SH3 also known as CD73). These cells are here envisioned to be used to differentiate into cardiomyocytes, cartilage cells, adipocytes, cells of osteogenic lineage as well as nerve cells (Weiss M L et al., Exp Neurol 182: 288, 2003; Fu Y S, et al., J Biomed Sci. 11: 652, 2004; Fu Y S et al., Stem Cells 24: 115, 2005; and Sarugaser R et al., Stem Cells 23: 220, 2005).

UCM cells of the Wharton's Jelly, like MSC, express intermediate levels of human leukocyte antigen (HLA) major histocompatibility complex (MHC) class I molecules and very low levels of (HLA) class II and Fas ligand; UCM cells do not express the co-stimulatory molecules B7-1, B7-2, CD40 or CD40L and are therefore not immunogeneic, as these co-stimulatory molecules are required for a full T-cell response. (Le Blanc K et al., Scand. J. Immunol. 57: 11, 2003; and Glennie S et al., Blood 105:2821, 2005).

Umbilical cord blood (UCB) is a viable source of hematopoietic stem cells for transplantation of children and adults undergoing treatment for hematological malignancies. However only 4% of adults 70 kg and over have a UCB unit available which contains the widely accepted minimum cell dose of 1.5×10⁷ mononuclear cells per kilogram. Co-transplantation of hematopoietic stem cells with mesenchymal stem cells may enhance engraftment and therefore decrease transplant-related morbidity and mortality from delayed leukocyte recovery associated with a low pre-transplant cell dose.

Umbilical cord matrix (UCM) cells, found in the Wharton's Jelly, were easily and reliably extracted from minced pieces of cord by culture in RPMI+20% fetal bovine serum at 37° C. and 5% humidified CO₂. It was observed that UCM cells best expanded in medium containing 20% FBS. This procedure can also be used to expand UCM cells in human serum, autologous serum, and the serum-free commercially available medium X-VIVO 10. Small (1-3 mm) minced pieces of umbilical cord can be cyropreserved at the time of delivery in 10% DMSO solution.

UCM cells exhibit a fibroblast morphology and express markers common to mesenchymal stem cells: CD73 (SH3), CD105 (SH2), CD 29, CD44, CD49b, CD117, CD166, STRO-1 and HLA-DR. UCM are negative for CD14, CD 19, CD34, and CD45. Morphology and cell surface marker expression is stable after greater than fifteen passages.

The present invention in certain embodiments provides methods and compositions for preparing CMSC in compliance with cGMP and cGTP conditions and practices, and materials that comply with the standards as regulated by the FDA, for use of these cells in humans for therapeutic purposes. The methods provided herein use cord blood serum or plasma of autologous origin, or use autologous serum or plasma, to add to the cells for culture or long term storage of CMSC. Prior art procedures have used serum or plasma from a non-human animal, or have used non-autologous serum or plasma (such as isologous, or allogeneic). However, use of animal serum or plasma is not ideal, for example, because of the possible presence of infectious particles.

The term “autologous” as used herein refers to materials that are taken from the same subject, for example, two or more biological samples taken from the same human.

The term “allogeneic” as used herein means materials taken from two different subjects of the same species, for example, two different human subjects, and generally assumes that the two subjects are genetically independent, i.e., are not identical twins or organismal clones.

The term, “xenogeneic” as used herein means materials taken from subjects of different species, for example, transfusion or implantation of material of porcine, bovine or canine origin into a species different than the source of the implant.

Allogeneic stem cell transplantation from a matched donor following myeloablative and non-myeloablative conditioning therapy has proven curative when used as part of a treatment for a number of inherited and acquired hematological disorders (Thomas E D, Int J Hematol 81: 89, 2005; and Resnick et al., Transpl Immunol 3: 207, 2005). The success of allogeneic transplantation is largely determined by compatibility between donor and recipient, which predicts the risk of severe and potentially fatal graft-versus-host disease.

About 75,000 cord blood units are stored in public banks. “The Stem Cell Therapeutic and Research Act” will allocate $79 million dollars for acquisition of 150,000 cord blood units that are believed to be necessary to broaden the donor pool to include recipients of all racial backgrounds and establish the “National Cell Transplantation Program” (Cord Blood-Establishing a National Hematopoietic Stem Cell Bank Program, The National Academies Press, Washington, D.C., 2005). Unfortunately, less than one third of patients needing an allogeneic transplant have a compatible donor available in their family. Registries have been established to match patients with compatible volunteer i.e. unrelated bone marrow/stem cell donors, but many patients, especially patients of non-Caucasian background, still lack stem cell donors. African-American and Asian donors are still underrepresented in existing bone marrow registries. Because of a lack of matched unrelated donors for minorities, the lead time necessary to acquire and process the hematopoietic stem cells from a volunteer, and the many advantages of UCB transplantation as listed above, continued advances related to UCB transplantation is needed to extend curative therapy to patients with hematologic malignancies and other hematologic disorders. Additionally, there can be a three to four month delay while the donor is contacted, tested, and arrangements for stem cell collections are made. Many patients cannot wait that long if their disease is progressing.

Further, general prior use of fetal bovine serum or plasma carries the risk of transmitting prion diseases and zoonoses, and xenogeneic proteins from an animal or allogeneic human serum or plasma may initiate immune responses in a subject. In addition to being a source of prions and other infectious particles, FCS is known to change the gene expression and functional characteristics of MSC (Shahdadfar A et al., Stem Cells 23: 1357, 2005). Alternative prior art procedures have used allogeneic human serum or plasma, however this material has been shown to be detrimental to the growth and function of CMSC. The present invention further provides methods and composition for the preparation of CMSC and cord blood cells and for subsequent long-term storage of these sources of stem cells obtained from the same donor in the same storage devise.

According to various embodiments of the methods provided herein, the sources of umbilical cord blood cells and CMSC are autologous, i.e. are obtained from the same donor. In certain embodiments, both the cells and CMSC are cryopreserved in the same container, for example in separate chambers of a multi-chamber container such as a freezer bag, using serum or plasma from the autologous cord donor for cryopreservation, generally admixed with a cryoprotectant. The container includes a mechanism such as a hermetically sealed plastic segment between each chamber of a bag. The plastic bridge between the chambers is large enough to allow opening, or even physical detachment, of a single storage chamber at any later time with continued cryopreservation of remaining chambers. Each chamber of the multi-chamber container also has a separate entry port.

In the methods provided herein, CMSC are extracted from the entire circumference of the umbilical cord of a mammal. The cord can first be divided into segments for storage and ease of manipulation. CMSC are prepared from each of a plurality of the short segments of the cord, by dissecting or mincing, i.e. dissecting each section of the umbilical tissue into small fragments, the umbilical tissue prior to cryopreservation.

Procedures for obtaining CMSC from the cord in the past have generally included mechanical extraction or enzymatic separation, following which cells are expanded in culture, for example, for several days, and are subsequently frozen for future use. However, ex vivo culture procedures used prior to cryopreservation carry a risk of contamination, and pose logistic problems, for example, a requirement that the cord blood and the umbilical cord arrive the same day for banking. Therefore a process or method that allows cryopreservation of fresh cord tissue would represent a significant improvement.

Prior attempts to freeze small segments of the cord have involved injecting cryoprotectant into the interior of the cord via a needle inserted into the cavity. However, recovery of the CMSC after thawing was observed to be minimal, and the yield and quality of the cells were highly variable. The cord segments obtained by this method also were not found to be suitable for storage in a bag or other standard container for long-term storage.

Prior art references relating to the collection and storage of cord cells have not addressed the issue of use of xenobiotic materials, and use of animal serum or plasma remains routine. Methods herein are advantageous in using chemicals and solutions that are well characterized and are prepared by methods approved by the FDA for use in humans.

### EXAMPLES

### Example 1

### Methods for Co-Transplantation of Human Umbilical Cord Matrix (UCM) Cells with Umbilical Cord Blood (UCB) Cells to Obtain Improved Speed of Engraftment

UCB are collected via cannulation of umbilical cord vessels at delivery. Mononuclear cells (MNCs) are isolated using Ficoll-Paque (Amersham Biosciences). Flow cytometry is performed on the MNCs to determine the number of CD34⁺ cells. The MNCs are stored in 10% dimethylsulfoxide (DMSO) in liquid nitrogen until ready for use. The UCB mononuclear cells are thawed, rinsed in fetal bovine serum (FBS) except as indicated below, and then resuspended in Dulbecco's Phosphate-Buffered Saline (PBS) prior to injection. Addition of FBS to buffers herein is according to conventional preparation of media, and is omitted in examples herein describing use of autologous cells, blood, and blood products.

Fresh umbilical cords are rinsed in saline and cut into pieces approximately one centimeter in length. The umbilical arteries and the umbilical vein are removed and the remaining tissue is placed in six well plates in RMPI plus 20% FBS and antibiotics (penicillin 100 μg/mL, streptomycin 10 μg/mL, amphotericin B 250 μg/mL) and incubated at 37° C. in 5% CO₂. UCM cells migrate from the cord and adhere to the plastic wells for about one week. The supernatant and the cord are discarded and cells are detached from the plate using 0.25% trypsin-EDTA (Invitrogen). UCM cells are expanded in plastic flasks using the aforementioned culture conditions. Flow cytometry is performed using CD73 (SH3), CD105 (SH2), CD 29, CD44, CD49b, CD14, CD34, CD45 as an assay for homogeneity.

Cells prepared as described above are injected via either of two different routes: intravenous (IV) or intra-bone marrow (IBM). Recipients are eight to ten week-old mice sublethally irradiated with 3.5 Gy from a ¹³⁷Cs source (2.115 Gy/min). Intravenous injection is via the lateral tail vein of mice. IBM injection is performed as described by Levac et al. (Levac, K et al., Exp Hematol. 33: 1417, 2005) as follows. Mice are anesthetized with an intraperitoneal injection of 0.015 mL/g body weight of a 2.5% solution of tribromoethanol. The right hind leg is shaved and disinfected. The knee is flexed to 90 degrees and a hole is drilled into the femur with a short 27-gauge needle attached to a 3-mL syringe filled with PBS. The first needle is removed and a 28-gauge needle with a 0.3 mL insulin syringe containing the cells is inserted into the femur. The cell dose injected for a total volume of 30-50 μL. The skin is closed with 6-0 vicryl suture (Ethicon).

The organ distribution of injected UCM cells after each mode of injection is determined by immunohistology of different target organs, including bone marrow, spleen, liver and lung. For identifying human UCM, human UCM cells that have been retrovirally transfected or marked with the green fluorescent protein (GFP) gene are used. The presence of GFP protein on GFP-tranduced cells in mouse tissue sections is assessed by assaying with a rabbit anti-GFP antibody. The human origin of these cells in mouse tissues is assessed by an antibody directed against human β₂-microglobulin.

Since GFP expression in tissue may be unstable, a second method to determine organ distribution of injected UCM cells is also used. Thus organ distribution is also determined by injecting male human UCM cells into female mice and assessing for presence of the human Y-chromosome by PCR.

The time points assessed after injection are each of 2 days, 7 days and 4 weeks. Since the IV infusion of UCM cells results in the majority of cells being sequestered in the lung and/or spleen before reaching the bone marrow, the UCM cells are also injected directly into bone as described above.

Initially, three different concentrations of UCB cells are injected to establish the length of time required for engraftment for varying cell doses, at each of IV and IBM routes of administration. A lower cell dose may be required for engraftment following IBM route of delivery. Twelve to twenty-four hours after irradiation, either 5×10⁵, 10⁶, or 5×10⁶ UCB cells resuspended in PBS are injected into the tail vein of mice.

Once the engraftment kinetics at each UCB concentration has been established, the dose that gives delayed engraftment is combined and co-injected with 10⁶ UCM. Control groups include mice receiving a dose of UCB cells that has been shown to establish delayed engraftment, a group that has been shown to provide timely engraftment, and a group that received PBS with no UCB cells. Engraftment is documented at 2, 3 and 4 weeks after cell infusion. Peripheral blood (50 μL) is collected from the submandibular plexus and a CBC is performed using the Hemavet 850 (CDC Technologies Inc. Oxford, Conn.). Further, the percentage of human CD45⁺ cells in murine blood is counted by flow cytometry. Differences in the human CD45 cell engraftment are determined by calculating the areas under the curves (AUCs) at each different time point. Mice are sacrificed after 6 weeks and bone marrow is collected by flushing both femurs and pelvis with RPMI medium. Single-cell suspensions from spleen, lung, and liver are prepared. The cell suspensions are stained with mouse anti-human monoclonal antibodies for flow cytometric analysis. PE or FITC-conjugated antibodies include monoclonal antibodies against CD45, CD34, CD 19, CD33, and CD38 (Becton-Dickinson).

Initial experiments are with autologous materials and recipients, i.e. UCM cells and UCB cells are from the same donor. As a control, allogeneic co-transplantation, i.e. UCB from one donor and UCM from another, is also performed.

### Example 2

### Transplantation of Varying Concentrations of Human UCB Cells to Determine Engraftment Delay

Initially human UCB cells at various doses are transplanted as the sole source of cells to determine cell doses that allow full engraftment and to determine a suboptimal cell concentration at which engraftment is delayed or will no longer occur. Two routes of injections are tested: intravenously and intra-bone marrow.

Mice are transplanted with UCB in order to establish engraftment kinetics. Mice in each of three experimental groups of mice are injected with either 5×10⁵, 10⁶, or 5×10⁶ cells. As controls, a single mouse is irradiated and receives a saline injection, and another single mouse is not irradiated and receives a saline injection. The experiment is done both IV and IBM, for example, a total of 28 mice in an experiment using four mice per experimental group. If all IBM mice have rapid engraftment at the lower dose, doses of 10⁵ cells or even 5×10⁴ cells are used with similar controls.

Statistical analyses are performed using table curve software (SPSS). The kinetics of human CD45 cell engraftment are evaluated by calculating areas under the curves (AUCs) at three time points (2 weeks, 4 weeks, 6 weeks). Differences in CD45⁺ cell AUCs between mouse groups are assessed using the Mann-Whitney rank sum test. Other statistics tests are performed using SPSS. When indicated, values are reported as mean±standard deviation (SD). Statistical significance is set for P<0.05. Following observing a positive effect on engraftment with autologous UCM, the experiments are repeated using allogeneic UCM cells.

### Example 3

### Analyzing Effect of Number of Autologous UCM Cells on Engraftment Rate in Co-Transplantation with UCB

Three groups of eight mice are injected with 5×10⁵, 10⁶, or 5×10⁶ UCB cells. For each cell dose, four mice are injected with 10⁶ UCM cells and four mice are not injected with UCM cells. Three controls are further performed at each UCB dose: an irradiated mouse that does not receive UCB and is injected with saline, a mouse that is not irradiated and receives saline, and a mouse that is irradiated and receives the UCM. The experiment is performed using each route of administration, both IV and IBM, and is also performed in an autologous fashion (UCM and UCB from same donor) and an allogeneic fashion (UCM and UCB from different donors), for a total of 132 mice.

Statistical analyses are performed using table curve software (SPSS). The kinetics of human CD45 cell engraftment are evaluated by calculating areas under the curves (AUCs) at different time points (2 weeks, 4 weeks, 6 weeks). Differences in CD45⁺ cell AUCs between mouse groups are assessed using the Mann-Whitney rank sum test. Other statistics tests are performed using SPSS. When indicated, values are reported as mean±standard deviation (SD). Statistical significance is set for P<0.05. If any positive effect on engraftment is seen with autologous UCM, the same experiments are conducted with allogeneic UCM cells.

### Example 4

### Affect of Co-Transplantation of UCB CD34⁺ Cells and Autologous UCM Cells on Engraftment In Vivo

UCM cells were grown in culture and were shown to produce more GM-CSF and G-CSF than similar numbers of adult bone marrow mesenchymal stem cells. The data showed that the UCM derived cells produced 178 pg/mL of GM-CSF compared to adult bone marrow mesenchymal stem cells, that produced 77 pg/mL; and the UCM derived cells produced 82.6 pg/mL G-CSF, compared to adult bone marrow cells that produced 7.9 pg/mL respectively.

Recipient mice of strain NOD/SCID were treated with anti-NK 1.1 antibodies, and were irradiated with 350 cGy. These were then injected with suboptimal (1×10⁴) numbers of cord blood CD34⁺ cells with and without 1×10⁶ autologous UCM cells, extracted from the same umbilical cord as the cord blood CD34⁺ cells. Bone marrow was harvested at six weeks post transplant from both femurs and tibias and peripheral blood was obtained via cardiac puncture. The percentage of human CD45⁺ cells in the bone marrow and the peripheral blood was assessed by flow cytometry. The data showed that control NOD/SCID mice transplanted with 1×10⁴ cord blood CD34⁺ cells alone had 3.0% human CD45⁺ cell engraftment in the bone marrow and 3.6% human CD45⁺ cells in the peripheral blood, while NOD/SCID mice transplanted with 1×10⁴ CD34⁺ cells and 1×10⁶ UCM cells had an average of 27.3% human CD45⁺ cell engraftment in the bone marrow and 3.9% human CD45⁺ cells in the peripheral blood. These results indicate that improved engraftment in vivo was observed with co-transplantation of suboptimal numbers of umbilical cord blood CD34⁺ cells and autologous umbilical cord matrix cells, compared to control transplantation of suboptimal numbers of umbilical cord CD34⁺ cells alone.

### Example 5

### Methods for Developing Conditions for Culture and Expansion of UCM Cells for Clinical Use

Human serum at different concentrations (5%, 10%, 20%) is tested with respect to its ability to support expansion of human UCM cells (autologous or isologous) in culture, and results are compared to FBS, at each of the concentrations, and as a control in absence of serum. Using current conventional methods, UCM cells are grown in RPMI 1640 as medium supplement. This example uses X-Vivo 10 (Cambrex Corporation) as a base medium instead of RPMI 1640, as X-Vivo 10 has been employed for studies with human cells and a drug master file for X-Vivo 10 is deposited with the FDA. Cell growth is evaluated and growth behaviors (cell count and doubling time) are evaluated daily, and the effect of different media on flowcytometric profile is analyzed.

The ability of the UCM cells to differentiate into bone tissue (osteogenesis assay) is used as a marker of intact and functional UCM cells. In this assay UCM cells for this test are contacted with medium containing dexamethasone (0.1 mM), L-ascorbic acid 2-phosphate 0.05 mM and beta-glycerol phosphate (3 mM). After 21 days the cells are fixed in 3.7% formaldehyde and then stained in 6% silver nitrate and exposed to UV light (20 min), and stained cells are counted.

Further, additives to the medium such as amino acids or epidermal growth factor (EGF), platelet derived growth factor (PDGF), leukemia inhibitory factor (LIF) or other growth factors are tested for obtaining optimum cell proliferation without the presence of FBS. Culture conditions that provide an aseptic closed system are used to reduce airborne contamination. Only cGMP and cGTP grade or appropriately qualified reagent such as trypsin and plastic ware are used. In order to make the process cGMP and cGTP compliant, a closed system with bags or a hollow fiber type bioreactor type system are used.

### Example 6

### Co-Injection of UCB and UCM Cells to Establish Engraftment in Larger Recipients

The examples herein are designed to show that co-injection of suboptimal numbers of UCB cells together with UCM cells can successfully establish engraftment, in larger recipients. For this purpose, recipients are transfused with only suboptimal amounts UCB, generally those units having MNC numbers of less than 1.5×10⁷/kg. Accelerated engraftment with optimal UCB numbers and the maintenance of functional characteristics of UCM cells after switching culture condition to cGMP and cGTP compliant conditions are also examined. Cells are manufactured by a facility in compliance with cGMP and cGTP, and further restricted to human MSC culture and expansion and designated by the NHLBI to provide such a cell therapy service to other clinical centers.

A phase I clinical trial is performed in which patients receive a standard cord blood transplant together with each of increasing numbers of UCM cells. Due to the current banking situation where no UCM are stored, the initial clinical trial uses allogeneic UCM cells. It is shown herein that sufficient number of cells are obtained from a small piece of cord and prepared and stored under cGMP conditions requiring minimal manipulation. Therefore it is contemplated herein that further clinical trials use autologous UCM cells.

In a clinical trial setting, three to four different dose levels of UCM cells are given along with UCB cells to cohorts of three patients in each group. The objective of the initial trial is to determine safety of the UCM infusions. The initial patients are those who receive a standard cord blood transplant with a sufficient number of UCB cells. Once this phase I trial is concluded, a phase II trial analyzes efficacy by transplanting a group of patients characterized in that only a suboptimal number of UCB cells are stored or are available (<1×10⁷ MNC/kg), and these patients are transplanted with the UCB together with a fixed dose of UCM cells.

### Example 7

### Methods for Preparation of CMSC in Autologous Cord Plasma or Serum for Long-Term Cryogenic Storage

Autologous cord blood serum or plasma is shown herein by the methods provided as useful for long-term storage of CMSC. The CMSC are obtained by different methods from the cord after delivery, for example, dissecting (mincing or cutting the cord) into fragments (small pieces) followed by addition of a cryoprotectant solution.

Alternatively to stored CMSC, a fresh supply of CMSC is obtained by mechanical or enzymatic extraction from the store or fresh cord and cultured for one or more days in serum free medium that is FDA approved to expand their numbers before being cryopreserved.

An exemplary cryoprotectant for use in the methods herein prepared as follows. Autologous cord blood plasma or serum (80-95%) that has been centrifuged is filtered through a 0.2 μm membrane and is mixed with dimethylsulfoxide (DMSO; 5-15%); and hydroxyethyl starch (HES; 3-8%). Because human serum or plasma obtained from an allogeneic donor causes significant changes in the pattern of gene expression in human matrix cells, affecting biological properties, the methods herein address that issue by using only autologous serum, individual plasma, or cord blood from the same source, i.e., from the same individual donor, for the purpose of preparing the cells for long-term storage.

The cord fragments or the isolated or cultured CMSC are frozen under controlled rate conditions, i.e., the external temperature is reduced systematically with specifically timed intervals of incubation at each lower temperature until the target freezing temperature is obtained.

### Example 8

### Storage of Cord Blood Stem Cells and CMSC in Separate Compartments of a Multi-Compartment Container

Autologous CMSC and cord blood cells, i.e. obtained from the same individual are stored in the same container at the time of banking to maximize convenience, and to avoid unwanted mixing and contamination.

The method of using a plastic bag for cryogenic storage having at least two chambers is suitable for cryopreserved minced cord fragments. Each chamber is accessible via a separate port and equipped with an identifier. The plurality of chambers allow storage of cord fragments, and/or stem cells from the autologous, same source or heterologous or allogeneic, from different sources of the same origin, in this case umbilical cord blood mononuclear cells and CMSC.

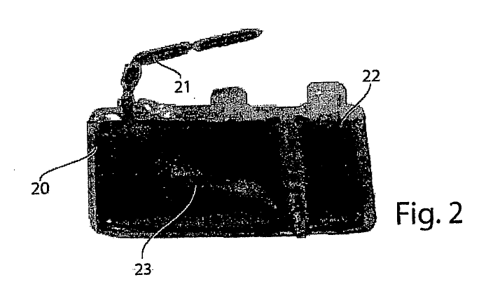

The storage container is a standard cryopreservation bag that however is separated into a number of discrete chambers. FIG. 2 shows a cryogenic bag 20 (Pall Corporation, East Hills, N.Y.) that can be used for long-term storage of cord blood cells and CMSC from the same donor. The bag 20 contains segments 21 that have patient-specific data engraved in the lining. The bag has a smaller compartment 22 and a larger compartment 23 for storing cord blood cells and CMSC. A solid plastic lining separates each chamber, each of which is individually removable and individually openable. A user thereby removes samples as needed by breaking away or cutting off one chamber, thereby processing only the amount of stem cells that are frozen in that particular chamber.

Alternatively, as each chamber is equipped with a corresponding separate entry port, the user accesses that discrete chamber. Each chamber further includes a patient/donor identifier and other relevant data attached to it and the identifier optionally includes additional information.

The methods herein can be used selectively to provide or remove one or a small number from among multiple iterations of chambers of frozen stem cells from the same donor. These are accessed for further cell manipulations including direct therapeutic administration, or alternatively, cell expansion, use as a feeder layer, or further culture to differentiate the cells into suitable transplants for various tissues, such culture including culture in the presence of well known differentiation factors such as epidermal growth factor (EGF), insulin-like growth factor (IGF) and keratinocyte growth factor (KGF).

An exemplary use of stem cells from the umbilical cord is support of a stem cell transplant. This use has in the past been limited, however, because the number of cord blood stem cells obtained and/or stored is frequently too low for larger recipients. Using the multi-compartment storage system in this situation, the CMSC from an additional chamber are thawed to use as a stromal (feeder) layer, to support cell number expansion by culture of cord blood stem cells.

The methods herein provide preparation of CMSC for cryopreservation that are performed under conditions that conform to FDA standards for current good manufacturing practices. This method involves using only reagents, plasticware and procedures that are approved for use with human cells. The methods provided herein do not use animal serum or plasma components or allogeneic serum or plasma from a corresponding mammalian individual. Such non-autologous components are known to be detrimental to the number and biological functions characteristic of CMSC.

### Example 9

### Method of Preparing a Segment of the Umbilical Cord for Long-Term Cryogenic Storage

A method was developed to prepare a small section of the cord (about 1 cm) with minimal manipulation compliant with cGMP conditions. An embodiment of the method herein includes, preparation of small cord segments, followed by dissecting, i.e. mincing, the small segment of the cord. This involves mincing of the cord with a scissor and freezing the small pieces in 10% DMSO and autologous (cord) plasma (to avoid exposure to allogeneic and/or animal serum or plasma). The entire circumference of the umbilical cord is utilized herein to obtain CMSC. After the cord is received from the donor, a plurality of small lengths is produced and each is subjected to a dissection or mincing process, resulting in a plurality of sets of fragments of sufficiently small size to result in even and consistent exposure of each of the CMSC fragments to cryoprotectant solution described herein or an equivalent. Reducing the fragment size was found to result in excellent recovery and yield of CMSC cells after thawing. This technique therefore fulfils the requirement for a clinical trial where we would need UCM cells from cords prepared under cGMP conditions.

The homogenization or mincing process was performed, for example, using an instrument in a plastic cartridge designed for single use. Alternatively the fragments were dissected manually, using, by way of example but not restricted to, a scissor, scalpel, disposable lancet, or other similar instrument. The fragments were then transferred to a container suitable for cryopreservation and long-term storage. The fragments containing CMSC were cryopreserved under controlled rate condition. After thawing, cells were expanded, by cell culture of the fragments, and the cells so obtained were found to express appropriate surface markers and display functional characteristics.

### Example 10

### Culture Methods and Biological Characteristics

Isolation of UCM cells from the Wharton Jelly was performed using the entire umbilical cord obtained from full-term deliveries. The cells were extracted and placed into 2 inch microtiter plates and kept at 37° C. (5% vol/vol CO₂) in media containing RPMI 1640/20% FCS. After two passages cells were transferred into flasks. Cultures without addition of cytokines were kept at 37° C. (5% vol/vol CO₂), and three fifths of the medium is renewed every 3 to 4 days. When grown to confluence, cells were detached with trypsin/EDTA and re-plated after washing, or cryopreserved in 10% dimethylsulfoxide, 25% FCS, and 65% RPMI medium.

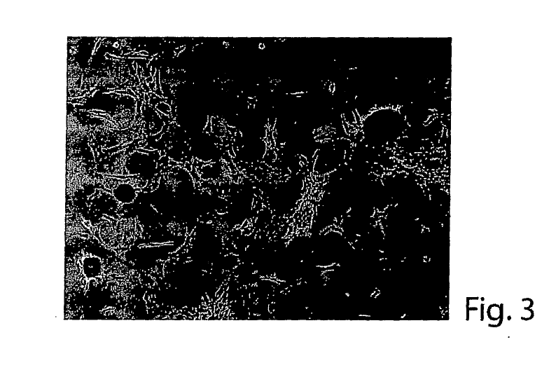

FIG. 3 shows a growth pattern of UCM cells inoculated into culture dishes containing RPMI growth medium/20% FCS. Culture-expanded UCM cells adhered to plastic and were found to have fibroblast like features, as shown in FIG. 3. The large dark spots represent areas of intense cell production.

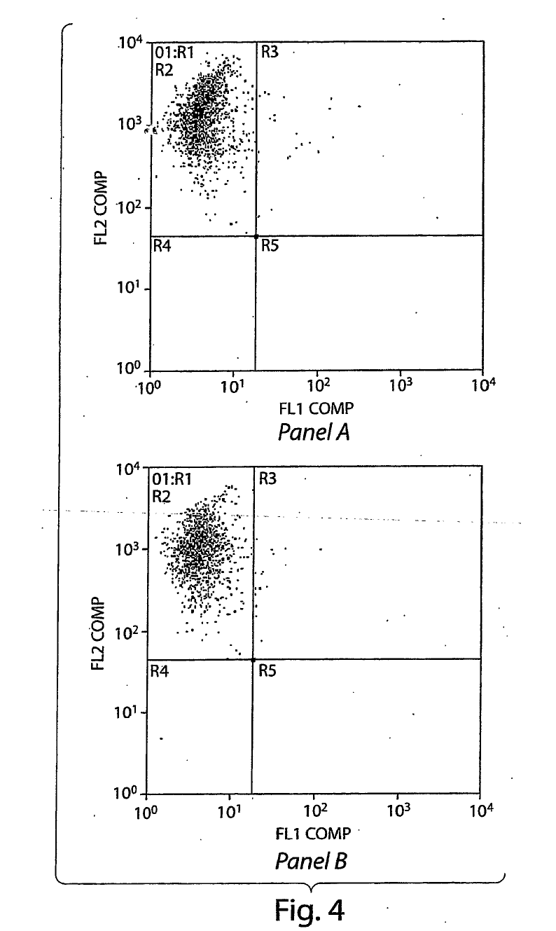

As shown by flow cytometry, cells were negative for the surface antigens CD34, CD45, CD14, CD40, CD80 and CD86. Thus these cells were found to be early stage stem cells, expressing the MSC markers CD73 and CD105. FIG. 4 shows a flow cytometric profile of UCM cells. In FIG. 4 Panel A, the abscissa represents the CD34 cells and the ordinate represents CD73. In FIG. 4 Panel B, the abscissa in represents the CD34 cells and the ordinate represents CD105 cells. The cells stained positive for CD105 (SH2), CD73 (SH3) and CD44. Further data indicated that the cells differentiated under appropriate conditions into adipocytes and osteoblasts.

Similar examples of isolation and characterization of UCM from Wharton's Jelly are shown using human serum in place of PCS.

### Example 11

### Culture Using Irradiated Feeder Cells

This example was performed using UCB cells that were expanded, by culture, on irradiated UCM feeder layers. CD34 cells were obtained from UCB using the Miltenyi immunomagnetic separation device (Miltenyi Biotec GmbH, Bergisch Gladbach, Germany). The CD34 enriched cells were plated on the irradiated UCM (autologous setting). Hematopoietic colonies were quantified after two weeks in medium containing methylcellulose. The data summarized below in Table 1 show that significantly more colonies were generated when an UCM feeder layer was present. CFU is mean number of hematopoietic colonies; CFU-E is number of erythrocyte CFUs; CFU-GEMM is number of granulocyte, erythrocyte, monocyte and megakaryocyte CFUs; and CFU-GM is granulocyte-macrophage CFUs. Analyses were performed on Day 14. The mean colony count of three examples is presented.

TABLE 1

| | UCB no UCM | UCB Plus UCM | Fold Increase |
|---|---|---|---|
| CFU-E | 55 | 137 | 2.49 |
| CFU-GEMM | 21 | 39 | 1.85 |
| CFU-GM | 120 | 242 | 2.01 |

### Example 12

### Method of Using Wharton's Jelly Cells as a Feeder Layer to Increase Number of Expanded Hematopoietic Cells

Umbilical cord matrix (UCM) was obtained as shown in Example 1 and other examples above. Natural killer (NK) cells were isolated from peripheral or cord blood using the Miltenyi immunomagnetic separation device (Miltenyi Biotec GmbH, Bergisch Gladbach, Germany).

Cells from Wharton's Jelly were obtained for use as a feeder layer by a procedure to collect Wharton's Jelly from the umbilical cord under sterile conditions as shown in U.S. patent application publication number 2003/0161818. The cord was cut transversely with a scalpel, and each section was transferred to a sterile container containing phosphate buffered saline (PBS) with CaCl₂ (0.1 g/l) and MgCl₂6H₂O (0.1 g/l) to remove surface blood from the section with gentle agitation. The section was then removed to a sterile surface, the outer layer of the section was incised along the longitudinal axis of the cord, and blood vessels of the umbilical cord (two veins and an artery) were removed by dissection. Wharton's Jelly was collected into a sterile container, or cut into smaller sections, of size such as 2-3 mm³ for culturing the included cells.

Genes for introduction into feeder layer cells were transfected into Wharton's Jelly cells by electroporation, a method well-known in the art, for example, Toneguzzo et al. 1986, PNAS 83:3496-3499. Viability of cells was assessed by flow cytometry using standard methods.

The NK cells were plated on the Wharton's Jelly cells in medium containing methylcellulose. Hematopoietic colonies were quantified after two weeks of culture. The number of expanded NK cells was found to have been increased when genetically manipulated Wharton's Jelly cells were used as the feeder layer, compared to NK cells cultured with control feeder cells not transfected, or feeder cells transfected with the vehicle vector only.

### Example 13

### Function of Natural Killer Cells Expanded with Feeder Cells for Use After Cord Blood Transplant

In treatment of certain diseases such as leukemia, bone marrow transplants are standard therapeutic procedures. Further, Natural Killer (NK) cell-mediated cytotoxicity can control the leukemia relapse, and protect the recipient patient from graft-versus-host disease (GVHD) that is observed to occur after allogeneic stem cell transplant.

Cord blood (CB) is rich in NK cells that have properties of proliferation and cytotoxicity similar to those of adult blood NK cells. Hence these cells are attractive for developing strategies to eliminate residual disease after cord blood transplant.

In this example, CB mononuclear cells were CD3 depleted and cells remaining were cryopreserved as described herein by immunomagnetic microbead selection (Miltenyi Biotec, Auburn, Calif.). Cells were thawed, and were plated for NK expansion with a feeder layer of irradiated umbilical cord mesenchymal (UCM) cells, the UCM having been obtained either from the same (autologous) or from an unrelated (allogeneic) cord donor, and having been cultured in presence or absence of each of IL-2 (1000 IU/ml), IL-15 (10 ng/ml), IL-3 (10 ng/ml) and Flt3 (10 ng/ml). Control NK cells were plated in the absence of feeder cells.

It was observed at a median of 19 days of culture (range 14-21 days), that there was a significantly greater extent of expansion (range 3.5-72 fold) of CD56⁺/CD3⁻ cells in cultures with the UCM feeder layer, and in the presence of cytokines, compared to controls (mean 21.2±20.8 fold increase, compared to 1.6±0.9 fold increase with feeder layer only, and 1.8 ±0.89 fold increase with cytokines only, p=0.039 and p=0.041 respectively). There was no significant difference observed in NK expansion between autologous and allogeneic UCM feeder layers (29.6±26.8 compared to 12.8±8.9 fold, p=0.243).

Expanded CB-NK cells were then tested for biological function viz., cytotoxicity using K562 cells. K562 cells are an established cell line that was derived from a patient having chronic myeloid leukemia, and a colorimetric assay with fluorescent dye PKH67-GL (Sigma, St. Louis, Mo.) was used to assess cytotoxic NK capability. CB-NK cells expanded by culture either with autologous or allogeneic UCM feeder layers were found to display enhanced cytotoxicity compared to controls plated with cytokines only (91.78±0.7% compared to 82.5±1.8%, p=0.003 and 89±2.3% compared to 83.7±0.18%, p=0.056, respectively).

In order to test whether expanded transfected CB-NK cells are useful for potentially targeting malignant cells, expanded CB-NK cells were electroporated, with mRNA transcribed from plasmid green fluorescent proetin (GFP) DNA by in vitro transcription. Flow cytometry was used to detect viability, which was 94%, 92% and 93% for non-transfected, GFP-DNA and GFP-mRNA samples respectively. GFP-mRNA expression at 24 hours was observed to be significantly higher (range 36.6-50.8%, mean 42.8±5.2%) compared to GFP-cDNA controls (mean 4.2±0.35%, p<0.001). Mean GFP-mRNA expression was 35%, 31% and 16.5% at 48, 72 and 144 hours respectively.

In summary, CB-NK cells were substantially expanded by culture with a feeder layer of UCM cells, and cytotoxicity was preserved. Further, the expanded cells were also capable of being genetically modified by transfection with mRNA of a gene of interest.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting. The contents of all references cited herein are incorporated by reference.

1. A method for preparing cord matrix stem cells (CMSC) for cryopreserving, the method comprising contacting the CMSC with a cryoprotectant and cord blood serum or plasma, wherein the serum or plasma is obtained from a source autologous in origin to the CMSC, and wherein the CMSC are isolated from a plurality of locations along an entire circumference of a transverse section of an umbilical cord.

2. (canceled)

3. The method according to claim 1 wherein the cryoprotectant is selected from the group consisting of dimethyl sulfoxide, glycerol, ethylene glycol, and propylene glycol.

4. The method according to claim 1 wherein the source is human.

5. The method according to claim 1 further comprising, after obtaining the CMSC from the source, cryopreserving the CMSC without culturing the cells to expand the cell number.

6. The method according to claim 1 further comprising, prior to cryopreserving, expanding the CMSC cell number by culturing.

7. The method according to claim 6 wherein expanding the CMSC comprises culturing the cells for at least one day.

8. The method according to claim 6 wherein expanding the CMSC comprises culturing the cells for at least two days.

9. The method according to claim 1 wherein obtaining the CMSC further comprises, prior to cryopreserving, dissecting the cord to obtained resulting fragments, and isolating the CMSC from the fragments.

10. The method according to claim 9, wherein prior to isolating the CMSC, the fragments are cryopreserved.

11. The method according to claim 1, further comprising contacting the cord, blood and/or plasma using sterile technique, sterile apparatus, and sterile buffers, wherein the buffers are adjusted to physiological pH and osmolarity.

12. A method of cryopreserving, separately or together, a plurality of types of stem cells from a subject, the method comprising apportioning the stem cells into a separate chamber of a container comprising a plurality of chambers, wherein each of the chambers is separately accessible.

13. The method according to claim 12, wherein the types of stem cells are obtained from sources selected from the group consisting of cord, matrix, placenta, cord matrix stem cells (CMSC) and blood cells.

14. The method according to claim 12, wherein each of the stem cells is separately cryopreserved in a chamber within the same container.

15. The method according to claim 12, wherein the container is a plastic bag and the separated chambers are separable compartments of the bag.

16. A method of preparing an umbilical cord obtained from an animal subject for cryopreservation, the method comprising:
preparing a plurality of segments of the cord;
dissecting each of the plurality of segments, wherein a plurality of resulting cord fragment preparations are obtained from each of the segments; and
cryopreserving separately each of the plurality of fragment preparations, wherein the umbilical cord is cryopreserved.

17. The method according to claim 15, wherein the segments are less than about 2 cm in length.

18. The method according to claim 15, wherein the segments are less than about 1 cm in length.

19. The method according to claim 15, further comprising after cryopreserving, isolating cord matrix stem cells (CMSC) from the fragments.

20. The method according to claim 15, further comprising prior to dissecting, contacting the cord with sterile plasticware or glassware, and sterile buffer of physiological pH and osmolarity.

21. The method according to claim 15, wherein the segments comprise all or a portion of a circumferential transverse section of the cord.

22. The method according to claim 15 wherein the source is a mammal.

23. A kit comprising a plurality of chambers wherein each chamber contains at least one cryopreserved material selected from the group of cord matrix stem cells (CMSC) and cord blood cells, wherein the CMSC and cord blood cells are obtained from an autologous source, wherein the chambers comprise separate compartments attached within a container, and each chamber is separably accessible so that within each chamber are provided independently with respect to the remainder of the chambers.

24. A kit comprising a plurality of chambers each chamber including cryopreserved cord matrix stem cells (CMSC) and/or cord blood cells, wherein the chambers comprise separate compartments attached within a plastic bag, wherein the CMSC and cord blood cells within each chamber are autologous, wherein each chamber is separately openable and wherein CMSC within each chamber are used independently with respect to the remainder of the chambers.

25. The kit according to claim 23, wherein each chamber contains a unit dose of CMSC.

26. The kit according to claim 24, wherein CMSC and/or cord blood cells in the plurality of chambers are from an autologous source.

27. A method of increasing the number of hematopoietic cells, the method comprising:
transfecting at least one gene into feeder cells thereby improving ability of the feeder cells to serve as a feeder layer; and
culturing the hematopoietic cells with the feeder layer, wherein the number of hematopoietic cells is increased.

28. The method of claim 27, wherein culturing further comprises using a blood product that is autologous to the hematopoietic cells or the feeder cells.

29. The method according to claim 27, wherein the gene encodes at least one protein selected from the group consisting of granulocyte-colony stimulating factor (G-CSF), granulocyte macrophage cell stimulating factor (GM-CSF), stem cell factor (SCF), thrombopoietin (TPO), erythropoietin (EPO), epidermal growth factor (EGF), keritinocyte growth factor (KGF), and other proteins that support the expansion and proliferation of cells.

30. The method according to claim 27, wherein the feeder cells are Wharton's Jelly cells.

31. The method according to claim 27, wherein the hematopoietic cells are CD34⁺ hematopoietic progenitor cells.

32. The method according to claim 31, further comprising culturing the CD34⁺ hematopoietic progenitor cell and developing the cells into least one cell type selected from the group consisting of natural killer cells, T cells, and dendritic cells.

33. The method of claim 27 wherein the hematopoietic cells and the feeder cells are autologous.

34. A method of preparing feeder cells, the method comprising:
genetically manipulating feeder cells, wherein the genetic manipulating results in improving an ability of the feeder cells to serve as a feeder layer.

35. The method according to claim 34, wherein the feeder cells are genetically manipulated by transfecting genes into the feeder cells encoding at least one of granulocyte-colony stimulating factor (G-CSF), granulocyte macrophage cell stimulating factor (GM-CSF), stem cell factor (SCF), thrombopoietin (TPO), erythropoietin (EPO), EGF, KGF, and other proteins that support the expansion and proliferation of cells.

36. The method according to claim 34, wherein prior to manipulating, the method comprises isolating the feeder cells from human umbilical cord.

37. The method according to claim 34, wherein isolating the feeder cells comprises obtaining Wharton's Jelly cells.
